(19) United States
(12) Patent Application Publication
(10) Pub. No.: US 2024/0331877 A1
(43) Pub. Date: Oct. 3, 2024

(54) PROGNOSTIC MODELS FOR PREDICTING FIBROSIS DEVELOPMENT

(71) Applicant: Hoffmann-La Roche Inc., Little Falls, NJ (US)

(72) Inventors: Julio HERNANDEZ SANCHEZ, Welwyn Garden city (GB); Andreas MAUNZ, Basel (CH); Siqing YU, Basel (CH); Beatriz GARCIA GARCIA, Basel (CH)

(21) Appl. No.: 18/743,437

(22) Filed: Jun. 14, 2024

Related U.S. Application Data
(63) Continuation of application No. PCT/US2022/081817, filed on Dec. 16, 2022.
(60) Provisional application No. 63/330,756, filed on Apr. 13, 2022.
(60) Provisional application No. 63/290,628, filed on Dec. 16, 2021.

(51) Int. Cl. G16H 50/30; G06T 7/00; G06T 7/10; G16H 10/20; G16H 10/60; G16H 20/10; G16H 50/20

(52) U.S. Cl. CPC G16H 50/30; G06T 7/0012; G06T 7/10; G16H 10/20; G16H 10/60; G16H 20/10; G16H 50/20; G06T 2207/10101; G06T 2207/30041

(57) ABSTRACT

A method and system for predicting fibrosis development. Optical coherence tomography (OCT) image data may be received for a retina of a subject with neovascular age-related macular degeneration (nAMD). The OCT image data is processed using a model system comprising a machine learning model to generate a prediction output. A final output is generated based on the prediction output in which the final output indicates a risk of developing fibrosis in the retina.

### CROSS-REFERENCE TO RELATED APPLICATIONS

This application is a continuation of International Application No. PCT/US2022/081817, filed on Dec. 16, 2022 and entitled “Prognostic Models for Predicting Fibrosis Development,” which claims priority to U.S. Provisional Patent Application No. 63/330,756, filed on Apr. 13, 2022 and entitled “Prognostic Models for Predicting Fibrosis Development,” and U.S. Provisional Patent Application No. 63/290,628, filed on Dec. 16, 2021 and entitled “Prognostic Models for Predicting Fibrosis Development,” each of which is incorporated herein by reference in its entirety.

### FIELD

The present disclosure relates generally to predicting fibrosis development and, more particularly, to methods and systems for automating the prediction of fibrosis development using machine learning.

### BACKGROUND

Age-related macular degeneration (AMD) remains the most frequent cause of irreversible blindness for people above 50 years old in the developed world. Neovascular AMD (nAMD) is an advanced form of AMD. The introduction of anti-vascular endothelial growth factor (anti-VEGF) therapies has significantly improved the prognosis of nAMD. However, a large proportion of patients suffer from irreversible vision loss despite treatment. In many instances, this vision loss is due to irreversible changes such as, for example, fibrosis development.

Fibrosis is thought to be a consequence of an aberrant wound healing process, which may be characterized by the deposition of collagen fibers that dramatically alter the structure and function of the different retinal layers. However, the pathophysiology of retinal fibrosis is complex and not fully understood, which has made developing specific therapies and identifying reliable biomarkers challenging. Currently available methods for detecting biomarkers that predict fibrosis development involve manual evaluation of images by human graders, making the detection less accurate, less efficient, and slower than desired.

### SUMMARY

In one or more embodiments, a method is provided predicting fibrosis development. Optical coherence tomography (OCT) image data may be received for a retina of a subject with neovascular age-related macular degeneration (nAMD). The OCT image data is processed using a model system comprising a machine learning model to generate a prediction output. A final output is generated based on the prediction output in which the final output indicates a risk of developing fibrosis in the retina.

In one or more embodiments, a method is provided for predicting fibrosis development. Optical coherence tomography (OCT) image data for a retina of a subject with neovascular age-related macular degeneration (nAMD) is received. The OCT image data is segmented using a segmentation model to generate segmented image data. The segmented image data is processed using a deep learning model to generate a prediction output. A final output is generated that indicates a risk of developing fibrosis in the retina based on the prediction output.

In one or more embodiments, a method is provided for predicting fibrosis development. At least one of clinical data or retinal feature data for a retina of a subject with neovascular age-related macular degeneration (nAMD) is received. The at least one of the clinical data or the retinal feature data is processed using a regression model to generate a prediction output. A final output that indicates a risk of developing fibrosis in the retina based on the prediction output is generated.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

### BRIEF DESCRIPTION OF THE DRAWINGS

The present disclosure is described in conjunction with the appended figures:

FIG. 1 is a block diagram of a prediction system 100 in accordance with one or more embodiments.

FIG. 2 is a block diagram of one example of an implementation for the model system from FIG. 1 in accordance with one or more embodiments.

FIG. 3 is a block diagram of one example of an implementation for the model system from FIG. 1 in accordance with one or more embodiments.

FIG. 4 is a flowchart of a process for predicting fibrosis development in accordance with one or more embodiments.

FIG. 5 is a flowchart of a process for predicting fibrosis development using OCT image data in accordance with one or more embodiments.

FIG. 6 is a flowchart of a process for predicting fibrosis development in accordance with one or more embodiments.

FIG. 7 is a flowchart of a process for predicting fibrosis development in accordance with one or more embodiments.

FIG. 8 is an OCT image in accordance with one or more embodiments.

FIG. 9 is a segmented image in accordance with one or more embodiments.

FIG. 10 is a table comparing the statistical results for feature-based models that use clinical data in accordance with one or more embodiments.

FIG. 11 is a table comparing the statistical results for feature-based models using retinal features derived from OCT image data in accordance with one or more embodiments.

FIG. 12 is a table comparing the statistical results for deep learning models using OCT image data and segmented image data in accordance with one or more embodiments.

FIG. 13 is a table comparing the statistical results for deep learning models using OCT image data and segmented image data in combination with clinical data in accordance with one or more embodiments.

FIG. 14 is a block diagram that illustrates a computer system in accordance with one or more embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

### I. Overview

The embodiments described herein recognize that it may be desirable to have methods and systems for predicting fibrosis development in neovascular age-related macular degeneration (nAMD) subjects that are less invasive, more efficient, and/or faster than currently available methods and systems. The development of fibrosis may include the onset of fibrosis and may include any continued fibrosis progression. Because fibrosis can lead to irreversible vision loss and because there is currently no treatment specifically targeted for fibrosis once it has developed, it may be important to predict if and when a subject being treated for, or who will be treated for, nAMD will develop fibrosis.

Typically, classic choroidal neovascularization (CNV) has been used as a prognostic biomarker for the development of fibrosis. CNV type and size are traditionally detected by manual observation of dye leakage in images generated via fluorescein angiography (FA), which may be also referred to as fundus fluorescein angiography (FFA). But FA (or FFA) imaging is invasive and using the images of such an imaging modality may be more burdensome than desired. For example, interpreting FA images to detect fibrosis currently relies on human graders with the requisite expertise or training.

Optical coherence tomography (OCT) imaging may be used to improve diagnosis and follow-up of patients with nAMD at risk for fibrosis because OCT imaging is less invasive. In addition to being less invasive, acquiring OCT images is easier as the technician training that may be needed is reduced. Further, OCT imaging may enable both qualitative and quantitative information to be obtained. Accordingly, the embodiments recognize that it may be desirable to have methods and systems for automating the prediction of fibrosis development via OCT images. Various morphological features found on OCT images have been associated with increased risk of fibrosis development, including, but not limited to: subretinal hyperreflective material (SHRM), foveal subretinal fluid (SRF), pigment epithelial detachment (PED), and foveal retinal thickness.

Thus, the embodiments described herein provide methods and systems for automating prediction of fibrosis development using OCT images and machine learning. The OCT images may be, for example, baseline OCT images. In one or more embodiments, deep learning models are used to process OCT images or segmented images (e.g., segmentation masks) developed from the OCT images to predict fibrosis. These segmented images may be generated using a trained deep learning model. These deep learning models may provide similar or improved accuracy for fibrosis prediction as compared to using the manual assessment of CNV type and size via FA images by human graders. Further, using these deep learning models to predict fibrosis may be easier, faster, and more efficient than using FA images or manual grading. Still further, using the deep learning models as described herein may enable improved fibrosis prediction in a manner that reduces the amount of computing resources needed.

In one or more embodiments, feature-based modeling is used to process retinal feature data extracted from segmented images to predict fibrosis. These segmented images may be generated from the same trained deep learning model as the segmented images discussed above with respect to the segmented images for the deep learning model approach. These feature-based models may provide similar or improved accuracy for fibrosis prediction as compared to using the manual assessment of CNV type and size via FA images by human graders. Further, using these feature-based models to predict fibrosis may be easier, faster, and more efficient than using FA images or manual grading. Still further, using the feature-based models as described herein may enable improved fibrosis prediction in a manner that reduces the amount of computing resources needed.

In some embodiments, clinical data may be used in addition to OCT image data, segmented image data, and/or the retinal feature data described above. This clinical data may be baseline clinical data that include values for various clinical variables such as, for example, but not limited to, age, visual acuity (e.g., a visual acuity measurement such as best corrected visual acuity measurement (BCVA)), or CNV type determined from FA images.

In various embodiments, machine learning models may process OCT images, segmented images, and/or the retinal feature data to detect the presence of CNV and classify CNV by its type. These machine learning models may detect the type of CNV with improved accuracy as compared to manual assessments of FA images via human graders. Further, using machine learning models to detect the type of CNV may reduce the amount of time and computing resources needed to detect the type of CNV.

Automated fibrosis detection using the machine learning-based methods and systems described herein may help guide prognosis and help in the development of new treatment strategies for nAMD and/or fibrosis. Further, automated fibrosis prediction may allow for better stratification and selection of subjects for clinical trials to ensure a richer and/or more accurate population selection for the clinical trials. Still further, automated fibrosis prediction may enable a more accurate evaluation of treatment response. For example, using machine learning models (e.g., deep learning and feature-based) such as those described herein to predict fibrosis development may help optimize the use of available medical resources and improve therapeutic efficacies, thereby improving overall subject (e.g., patient) healthcare.

Recognizing and taking into account the importance and utility of a methodology and system that can provide the improvements described above, the embodiments described herein provide machine learning models for improving the accuracy, speed, efficiency, and ease of predicting fibrosis development in subjects diagnosed with and/or being treated for nAMD. Further, the methods and systems described herein may enable a less invasive way of predicting fibrosis development, while also reducing the level of expertise or expert training needed for performing the prediction.

### II. Exemplary System for Predicting Fibrosis Development in nAMD

### II.A. Overview of System

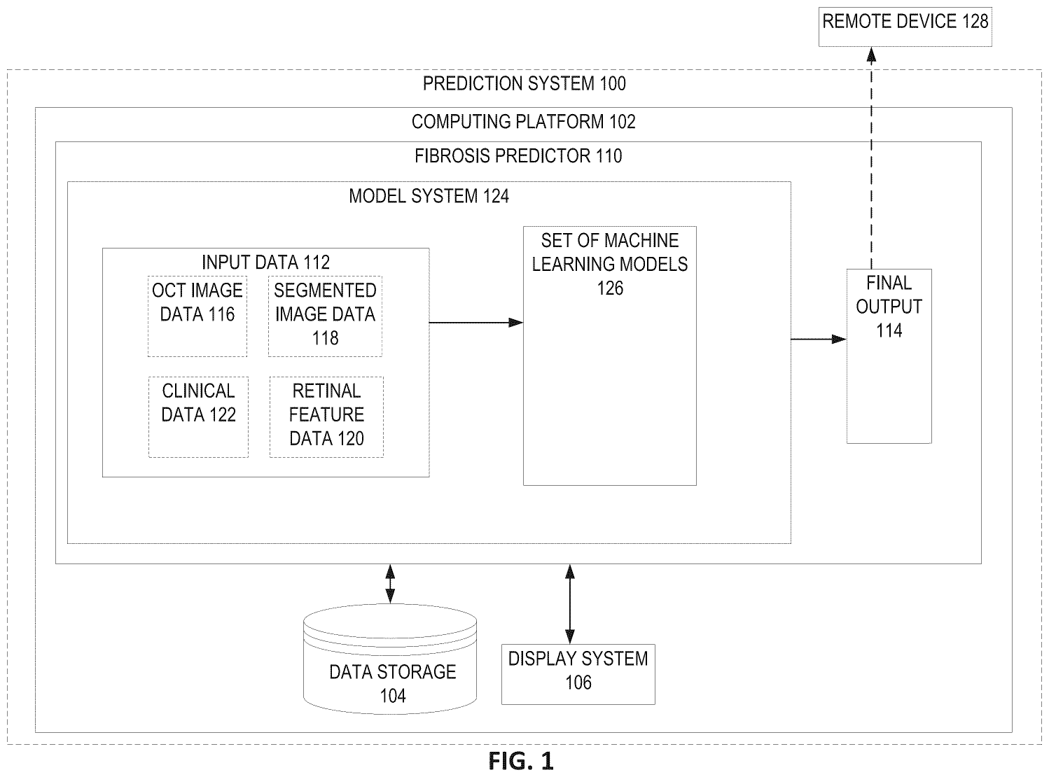

Referring now to the figures, FIG. 1 is a block diagram of a prediction system 100 in accordance with one or more embodiments. Prediction system 100 may be used to predict the development of fibrosis in the eye of a subject diagnosed with neovascular age-related macular degeneration (nAMD). In one or more embodiments, prediction system 100 includes computing platform 102, data storage 104, and display system 106. Computing platform 102 may take various forms. In one or more embodiments, computing platform 102 includes a single computer (or computer system) or multiple computers in communication with each other. In other examples, computing platform 102 takes the form of a cloud computing platform, a mobile computing platform (e.g., a smartphone, a tablet, etc.), or a combination thereof.

Data storage 104 and display system 106 are each in communication with computing platform 102. In some examples, data storage 104, display system 106, or both may be considered part of or otherwise integrated with computing platform 102. Thus, in some examples, computing platform 102, data storage 104, and display system 106 may be separate components in communication with each other, but in other examples, some combination of these components may be integrated together.

Prediction system 100 includes fibrosis predictor 110, which may be implemented using hardware, software, firmware, or a combination thereof. In one or more embodiments, fibrosis predictor 110 is implemented in computing platform 102.

Fibrosis predictor 110 receives and processes input data 112 to generate final output 114. Final output 114 may be, for example, a binary classification that indicates whether fibrosis development is predicted or not. This indication may be with respect to a risk of developing fibrosis. For example, the binary classification may be a positive or negative prediction for fibrosis development or may be a high-risk or low-risk prediction. This prediction may be made for a future point in time (e.g., 1 month, 2 months, 3 months, 4, months, 6 months, 8 months, 12 months, 15 months, 24 months, etc. after a first dose or more recent dose of treatment) or for an unspecified period of time. In other examples, final output 114 may be a score that is indicative of whether fibrosis development is predicted or not. For example, a score at or above a selected threshold (e.g., a threshold between 0.4 and 0.9) may indicate a positive prediction for fibrosis development, while a score below the selected threshold may indicate a negative prediction. In some cases, the score may be a probability value or likelihood value that fibrosis will develop.

Input data 112 may be data for a subject who has been diagnosed with nAMD. The subject may have been previously treated with an nAMD treatment (e.g., an anti-VEGF therapy such as ranibizumab; an antibody therapy such as faricimab, or some other type of treatment). In other embodiments, the subject may be treatment naïve.

Input data 112 may include, for example, without limitation, at least one of optical coherence tomography (OCT) image data 116, segmented image data 118, retinal feature data 120, clinical data 122, or a combination thereof. In one or more embodiments, input data 112 includes at least one of optical coherence tomography (OCT) image data 116, segmented image data 118, or retinal feature data 120 and optionally, includes clinical data 122.

OCT image data 116 may include, for example, one or more raw OCT images that have either not been preprocessed or one or more OCT images that have been preprocessed using one or more standardization or normalization procedures. An OCT image may take the form of, but is not limited to, a time domain optical coherence tomography (TD-OCT) image, a spectral domain optical coherence tomography (SD-OCT) image, a two-dimensional OCT image, a three-dimensional OCT image, an OCT angiography (OCT-A) image, or a combination thereof. Although SD-OCT, also known as Fourier domain OCT, may be referred to with respect to the embodiments described herein, other types of OCT images are also contemplated for use with the methodologies and systems described herein. Thus, the description of embodiments with respect to images, image types, and techniques provides merely non-limiting examples of such images, image types, and techniques.

Segmented image data 118 may include one or more segmented images that have been generated via retinal segmentation. Retinal segmentation includes the detection and identification of one or more retinal (e.g., retina-associated) elements in a retinal image. A segmented image identifies one or more retinal (e.g., retina-associated) elements on the segmented image using one or more graphical indicators. The segmented image may be a representation of an OCT image that identifies the one or more retinal elements or may be an OCT image on which the one or more retinal elements have been identified.

For example, one or more color indicators, shape indicators, pattern indicators, shading indicators, lines, curves, markers, labels, tags, text features, other types of graphical indicators, or a combination thereof may be used to identify the portion(s) (e.g., by pixel) of the image that have been identified as a retinal element. As one specific example, a group of pixels may be identified as capturing a particular retinal fluid (e.g., intraretinal fluid or subretinal fluid). A segmented image may identify this group of pixels using a color indicator. For example, each pixel of the group of pixels may be assigned a color that is unique to the particular retinal fluid and thereby assigns each pixel to the particular retinal fluid. As another example, the segmented image may identify the group of pixels by applying a patterned region or shape (continuous or discontinuous) over the group of pixels.

A retinal element may be comprised of at least one of a retinal layer element or a retinal pathological element. Detection and identification of one or more retinal layer elements may be referred to as layer element (or retinal layer element) segmentation. Detection and identification of one or more retinal pathological elements may be referred to as pathological element (or retinal pathological element) segmentation.

A retinal layer element may be, for example, a retinal layer or a boundary associated with a retinal layer. Examples of retinal layers include, but are not limited to, the internal limiting membrane (ILM) layer, the retinal nerve fiber layer, the ganglion cell layer, the inner plexiform layer, the inner nuclear layer, the outer plexiform layer, the outer nuclear layer, the external limiting membrane (ELM) layer, the photoreceptor layer(s), the retinal pigment epithelial (RPE) layer, an RPE detachment, the Bruch's membrane (BM) layer, the choriocapillaris layer, the choroidal stroma layer, the ellipsoid zone (EZ), and other types of retinal layer. In some cases, a retinal layer may be comprised of one or more layers. As one example, a retinal layer may be the interface between an outer plexiform layer and Henle's fiber layer (OPL-HFL). A boundary associated with a retinal layer may be, for example, an inner boundary of the retinal layer, an outer boundary of the retinal layer, a boundary associated with a pathological feature of the retinal layer (e.g., an inner or outer boundary of detachment of the retinal layer), or some other type of boundary. For example, a boundary may be an inner boundary of an RPE (IB-RPE) detachment layer, an outer boundary of the RPE (OB-RPE) detachment layer, or another type of boundary.

A retinal pathological element may include, for example, fluid (e.g., a fluid pocket), cells, solid material, or a combination thereof that evidences a retinal pathology (e.g., disease or condition such as AMD or diabetic macular edema). For example, the presence of certain retinal fluids may be a sign of nAMD. Examples of retinal pathological elements include, but are not limited to, intraretinal fluid (IRF), subretinal fluid (SRF), fluid associated with pigment epithelial detachment (PED), hyperreflective material (HRM), subretinal hyperreflective material (SHRM), intraretinal hyperreflective material (IHRM), hyperreflective foci (HRF), a retinal fluid pocket, drusen, and fibrosis. In some cases, a retinal pathological element may be a disruption (e.g., discontinuity, delamination, loss, etc.) of a retinal layer or retinal zone. For example, the disruption may be of the ellipsoid zone, of the ELM, of the RPE, or of another layer or zone. The disruption may represent damage to or loss of cells (e.g., photoreceptors) in the area of the disruption. In some examples, a retinal pathological element may be clear IRF, turbid IRF, clear SRF, turbid SRF, some other type of clear retinal fluid, some other type of turbid retinal fluid, or a combination thereof.

In one or more embodiments, segmented image data 118 may have been generated via a deep learning model. The deep learning model may be comprised of a convolutional neural network system that is comprised of one or more neural networks. Each of or at least one of these one or more neural networks may itself be a convolutional neural network.

Retinal feature data 120 may include, for example, without limitation, feature data extracted from segmented image data 118. For example, feature data may be extracted for one or more retinal elements identified in segmented image data 118. This feature data may include values for any number of or combination of features (e.g., quantitative features). These features may include pathology-related features, layer-related volume features, layer-related thickness features, or a combination thereof. Examples of features include, but are not limited to, a maximum retinal layer thickness, a minimum retinal layer thickness, an average retinal layer thickness, a maximum height of a boundary associated with a retinal layer, a volume of a retinal fluid pocket, a length of a fluid pocket, a width of a fluid pocket, a number of retinal fluid pockets, and a number of hyperreflective foci. Thus, at least some of the features may be volumetric features. For example, the feature data may be derived for each selected OCT image (e.g., single OCT B-scan) and then combined to form volume-wide values. In one or more embodiments, between 1 to 200 features may be included in retinal feature data 120.

Clinical data 122 may include, for example, without limitation, age, a visual acuity measurement, a choroidal neovascularization (CNV) type, or a combination thereof. The visual acuity measurement may be, for example, a best corrected visual acuity (BCVA) measurement. The CNV type may be an identification of type based on the assessment of fluorescein angiography (FA) image data. The CNV type may be, for example, occult CNV, predominantly classic CNV, minimally classic CNV, or Retinal Angiomatous Proliferation (RAP). In some cases, “classic CNV” may be used as the CNV type that captures both predominantly classic CNV or minimally classic CNV. In some cases, CNV type is identified based on a numbering scheme (e.g., Type 1 referring to occult CNV, Type 2 referring to classic CNV, and Type 3 referring to RAP). In one or more embodiments, at least a portion of clinical data 122 may be for a baseline point in time. For example, CNV type and/or BCVA may be obtained for the baseline point in time. The baseline point in time may be a time after nAMD diagnosis but just prior to treatment (e.g., prior to a first dose), a time period after the first dose of treatment (e.g., 6 months, 9 months, 12 months, 15 months, etc. after the first dose), or another type of baseline point in time.

Fibrosis predictor 110 uses model system 124 to process input data 112, which may include any one or more of the different types of data described above, and generate final output 114. Model system 124 may be implemented using different types of architectures. Model system 124 may include set of machine learning models 126. One or more of set of machine learning models 126 may receive input data 112 (e.g., some or all of input data 112) for processing. The data included in input data 112 may vary based on the type of architecture used for model system 124. Examples of the different types of architectures that may be used for model system 124 and the different types of data that may be included in input data 112 are described in greater detail below in Sections II.B. and II.C.

In one or more embodiments, final output 114 may include other types of information. For example, in some cases, final output 114 may include a clinical trial recommendation, a treatment recommendation, or both. A clinical trial recommendation may be a recommendation to include or exclude the subject from a clinical trial. A treatment recommendation may be a recommendation to change a type of treatment, adjust a treatment regimen (e.g., injection frequency, dosage, etc.), or both.

At least a portion of final output 114 or a graphical representation of at least a portion of final output 114 may be displayed on display system 106. In some embodiments, at least a portion of final output 114 or a graphical representation of at least a portion of final output 114 is sent to remote device 128 (e.g., a mobile device, a laptop, a server, a cloud, etc.).

### II.B. Fibrosis Predictor Using Deep Learning Model

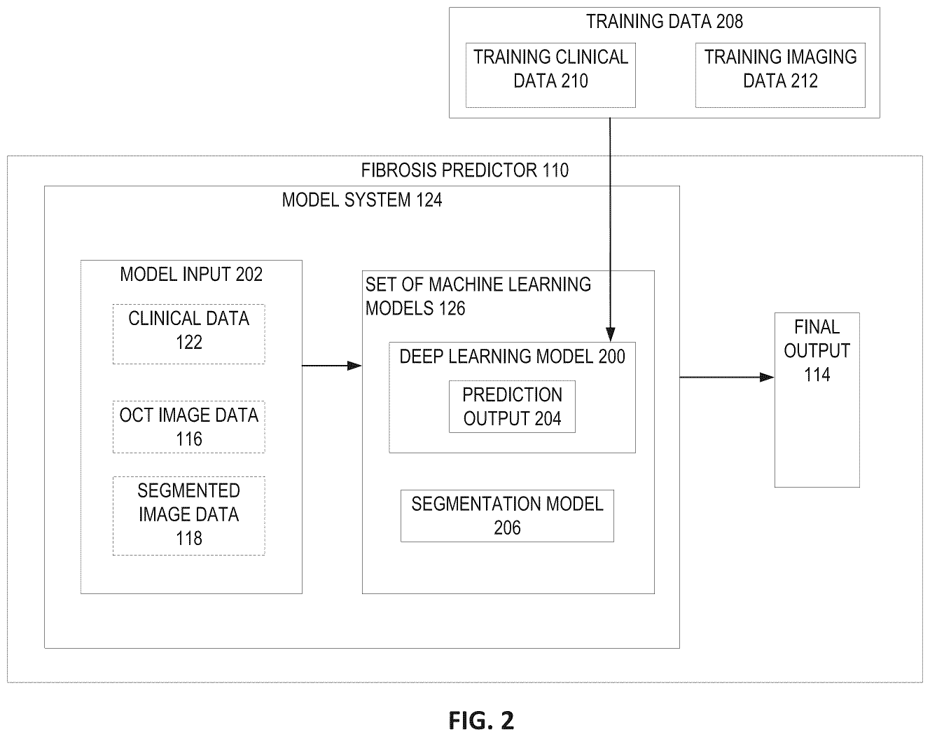

FIG. 2 is a block diagram of one example of an implementation for model system 124 from FIG. 1 in accordance with one or more embodiments. Model system 124 in FIG. 2 is described with continuing reference to FIG. 1. Model system 124 includes deep learning model 200, which may be one example of an implementation for a machine learning model in set of machine learning models 126. Deep learning model 200 may receive model input 202 and generate prediction output 204.

In one or more embodiments, model input 202 is formed using at least a portion of input data 112 described above with respect to FIG. 1. In some embodiments, model input 202 includes OCT image data 116. In other embodiments, model input 202 includes OCT image data 116 and at least a portion of clinical data 122 (e.g., a baseline CNV type, a baseline visual acuity measurement, age, or a combination thereof).

In some embodiments, model input 202 includes segmented image data 118. In other embodiments, model input 202 includes segmented image data 118 and at least a portion of clinical data 122 (e.g., a baseline CNV type, a baseline visual acuity measurement, age, or a combination thereof).

Deep learning model 200 may be implemented using a binary classification model. In one or more embodiments, deep learning model 200 is implemented using a convolutional neural network system that may be comprised of one or more neural networks. Each of or at least one of these one or more neural networks may itself be a convolutional neural network. In some embodiments, deep learning model 200 is implemented using a ResNet-50 model, which is a convolutional neural network that is 50 layers deep, or a modified form of ResNet-50.

When model input 202 comprises at least a portion of clinical data 122 in addition to either OCT image data 116 or segmented image data 118, deep learning model 200 may use a modified form of a convolutional neural network to concatenate vectors for the clinical data (clinical variables) to the OCT image data 116 or segmented image data 118, respectively. As one example, when deep learning model 200 is implemented using ResNet-50, a first portion of deep learning model 200 includes the ResNet-50 without its top layers. This first portion of deep learning model 200 is used to generate a first intermediate output based on the OCT image data 116 or segmented image data 118. A second portion of the deep learning model (e.g., the replacement for the top layers of the ResNet-50) may include a custom dense layer portion (e.g., one or more dense layers). A set of vectors for the clinical variables (e.g., baseline CNV type, baseline visual acuity, and/or baseline age) are concatenated to the first intermediate output generated by the first portion of the deep learning model 200 to form a second intermediate output. The second intermediate output is sent into the custom dense layer portion of deep learning model 200. In some cases, the output of the ResNet-50 in the first portion of deep learning model 200 may pass through an average pooling layer to form the first intermediate output.

Deep learning model 200 outputs prediction output 204 based on model input 202. Fibrosis predictor 110 may form final output 114 using prediction output 204. For example, prediction output 204 may be the likelihood that the eye of a subject diagnosed with nAMD will develop fibrosis. In one or more embodiments, prediction output 204 is a binary classification that indicates whether fibrosis development is predicted or not. In such examples, final output 114 may include prediction output 204. In other embodiments, prediction output 204 takes the form of a score (e.g., a probability distribution value or likelihood value) that is indicative of whether fibrosis development is predicted or not. In such examples, final output 114 may include prediction output 204 and/or a binary classification formed based on the score. For example, fibrosis predictor 110 may generate final output 114 as a binary classification or indication based on whether the score generated by the deep learning model is above a selected threshold (e.g., a threshold between 0.4 and 0.9).

In some embodiments, model system 124 may further include a segmentation model 206. Segmentation model 206 may receive OCT image data 116 as input and may generate segmented image data, such as segmented image data 118. Segmentation model 206 is used to automate the segmentation of OCT image data 116. Segmentation model 206 may include, for example, without limitation, a deep learning model. Segmentation model 206 may include, for example, one or more neural networks. In one or more embodiments, segmentation model 206 takes the form of a U-Net.

Deep learning model 200 may be trained using training data 208 for subjects diagnosed with and being treated for nAMD. Training data 208 may include training clinical data 210 and training image data 212. The training image data 212 may include or be generated from OCT images at a future point in time after the beginning of treatment. For example, the OCT images may have been generated at the 6-month interval, 9-month interval, 12-month interval, 24-month interval, or some other time interval after the beginning of treatment. Fibrosis development at this future point in time may be assessed by human graders.

### II.C. Fibrosis Predictor Using Feature-Based Model

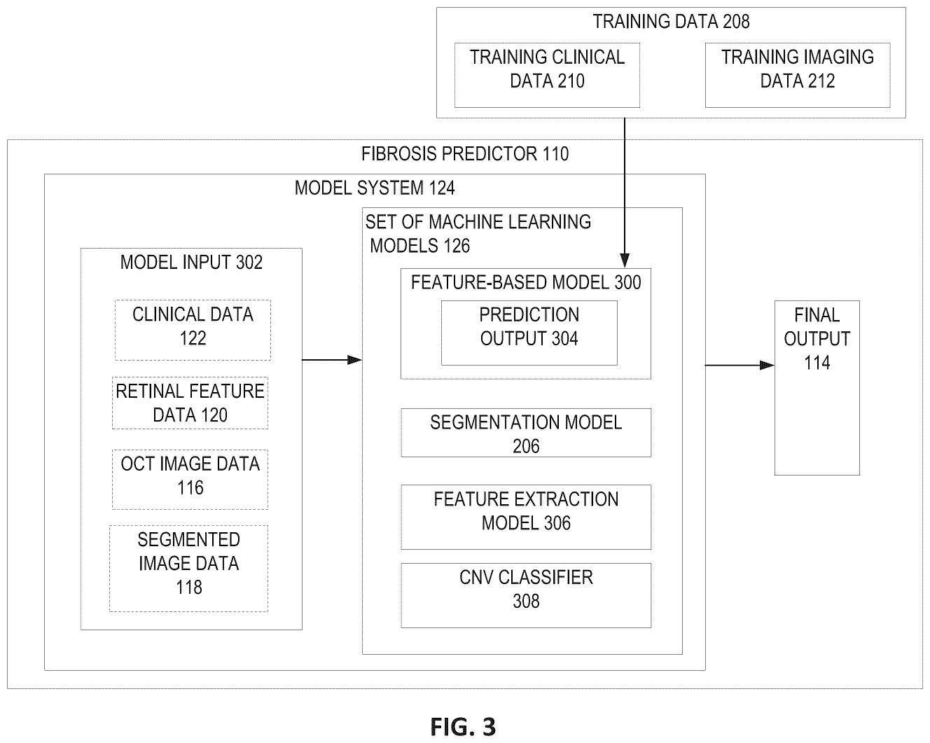

FIG. 3 is a block diagram of one example of an implementation for model system 124 from FIG. 1 in accordance with one or more embodiments. Model system 124 in FIG. 3 is described with continuing reference to FIGS. 1 and 2. Model system 124 includes feature-based model 300, which may be one example of an implementation for a machine learning model in set of machine learning models 126. Feature-based model 300 may receive model input 302 and generate prediction output 304.

In one or more embodiments, model input 302 is formed using a portion of input data 112 described above with respect to FIG. 1. For example, model input 302 includes retinal feature data 120. In other embodiments, model input 302 includes retinal feature data 120 and at least a portion of clinical data 122 (e.g., a baseline CNV type, a baseline visual acuity measurement, age, or a combination thereof). In still other embodiments, model input 302 includes at least a portion of clinical data 122 including a baseline CNV type, as well as baseline visual acuity measurement, age, or both.

Feature-based model 300 may be a regression model (or algorithm). For example, feature-based model 300 may be a logistic regression model, a linear regression model, or some other type of regression model. Feature-based model 300 may generate prediction output 304 in the form of a score (e.g., probability value or likelihood value). A score over a selected threshold (e.g., 0.5, 0.6, 0.7, or some other value between 0.4 and 0.9) may be a score that positively indicates fibrosis development. A score below this selected threshold may indicate that fibrosis is not predicted to develop.

In one or more embodiments, feature-based model 300 may be a regression model that is trained using one or more regularization techniques to reduce overfitting. These regularization techniques may include Ridge regularization, Lasso regularization, Elastic Net regularization, or a combination thereof. For example, the number of features used in feature-based model may be reduced to those having above-threshold importance to prediction output 304. In some cases, this type of training may simplify the feature-based model 300 and allow for shorter runtimes. For example, a Lasso regularization technique may be used to reduce the number of features used in the regression model and/or identify important features (e.g., those features having the most importance to the prediction generated by the regression model). An Elastic Net regularization technique depends on both the amount of total regularization (lambda) and the mixture of Lasso and Ridge regularizations (alpha). The cross-validation strategy may include a 5-fold or 10-fold cross validation strategy. The parameters alpha and lambda that minimize cross-validated deviance may be selected.

In one or more embodiments, model input 302 includes three baseline clinical variables from clinical data 122 including CNV type, BCVA, and age. In one or more embodiments, model input 302 includes, for each of the 1 mm and 3 mm foveal areas, SHRM grade (e.g., graded according to a centralized grading protocol), PED grade (e.g., graded according to a centralized grading protocol), and the maximal height of SRF. In one or more embodiments, model input 302 includes the maximal thickness between the OPL-HFL layer and the RPE, the thickness of the entire neuroretina from the ILM layer to the RPE layer, or both. In one or more embodiments, model input 302 includes baseline CNV type, baseline age, and baseline BCVA from clinical data 122 and central retinal thickness (CRT), subfoveal choroidal thickness (SFCT), a grade for PED, a maximal height of SRF, and a grade for SHRM from retinal feature data 120. In other embodiments, model input 302 includes CRT, SFCT, PED, SRF, and SHRM.

In some embodiments, model system 124 includes segmentation model 206, feature extraction model 306, or both. Segmentation model 206 may be the same pretrained model as described in FIG. 2. Segmentation model 206 may be used to generate segmented image data 118 from OCT image data 116 provided in model input 302. Feature extraction model 306, which may be one example of an implementation for a machine learning model in set of machine learning models 126, may be used to generate retinal feature data 120 based on segmented image data 118 included in model input 302 or segmented image data 118 generated by segmentation model 206.

In one or more embodiments, CNV type may be a type of feature included in retinal feature data 120. For example, CNV type may be determined by feature extraction model 306. In other embodiments, model system 124 includes CNV classifier 308. CNV classifier 308 may be one example of an implementation for a machine learning model in set of machine learning models 126. For example, CNV classifier 308 may include a machine learning model (e.g., deep learning model comprising one or more neural networks) that is able to detect a CNV type using OCT image data 116 instead of FA images. This CNV type may be referred to as a model-generated CNV or an OCT-based CNV type. In some cases, this CNV type is sent directly from CNV classifier 308 to feature-based model 300 for processing.

Feature-based model 300 outputs prediction output 304 based on model input 302. Fibrosis predictor 110 may form final output 114 using prediction output 304. For example, prediction output 304 may be the likelihood that the eye of a subject diagnosed with nAMD will develop fibrosis. In one or more embodiments, prediction output 304 is a binary classification that indicates whether fibrosis development is predicted or not. In such examples, final output 114 may include prediction output 304. In other embodiments, prediction output 304 takes the form of a score (e.g., a probability distribution value or likelihood value) that is indicative of whether fibrosis development is predicted or not. In such examples, final output 114 may include prediction output 304 and/or a binary classification formed based on the score. For example, fibrosis predictor 110 may generate final output 114 as a binary classification or indication based on whether the score generated by the deep learning model is above a selected threshold (e.g., a threshold between 0.4 and 0.9).

Feature-based model 300 may be trained using training data 208 for subjects diagnosed with and being treated for nAMD. Training data 208 may include the same training data as described with respect to FIG. 2.

### III. Exemplary Methodologies for Predicting Fibrosis Development

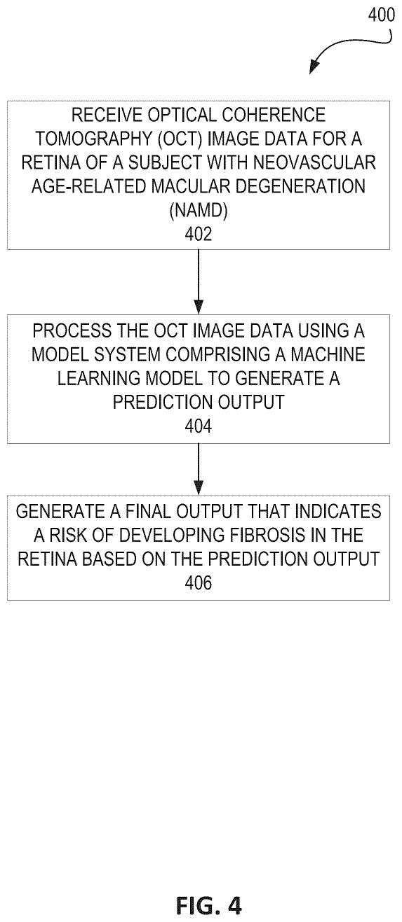

FIG. 4 is a flowchart of a process 400 for predicting fibrosis development in accordance with one or more embodiments. In one or more embodiments, process 400 may be implemented using prediction system 100 described in FIG. 1 and/or fibrosis predictor 110 described in FIGS. 1-3. Process 400 includes various steps and may be described with continuing reference to FIGS. 1-3. One or more steps that are not expressly illustrated in FIG. 4 may be included before, after, in between, or as part of the steps of process 400. In some embodiments, process 400 may begin with step 402.

Step 402 includes receiving optical coherence tomography (OCT) image data for a retina of a subject with neovascular age-related macular degeneration (nAMD). The OCT image data may be, for example, OCT image data 116 described with respect to FIGS. 1-3.

Step 404 includes processing the OCT image data using a model system comprising a machine learning model to generate a prediction output. The model system may be, for example, model system 124 described with respect to FIGS. 1-3. The machine learning model may include, for example, deep learning model 200 in FIG. 2 or feature-based model 300 in FIG. 3. In some cases, the model system includes a segmentation model (e.g., segmentation model 206 in FIGS. 2-3. In some cases, the model system includes a feature extraction model (e.g., feature extraction model 306 in FIG. 3). In some cases, the model system includes a CNV classifier (e.g., CNV classifier 308 in FIG. 3).

The prediction output generated in step 404 may be, for example, prediction output 204 in FIG. 2 or prediction output 304 in FIG. 3. The prediction output may be the likelihood that the retina of a subject diagnosed with nAMD will develop fibrosis. In one or more embodiments, the prediction output is a binary classification that indicates whether fibrosis development is predicted or not. For example, the binary classification may indicate whether the risk of fibrosis development is low or high. The prediction output may take the form of a score (e.g., a probability distribution value or likelihood value) that is indicative of whether fibrosis development is predicted or not.

The processing in step 404 may be performed in various ways. In one or more embodiments, the machine learning comprises a deep learning model (e.g., at least one neural network such as a convolutional neural network). The deep learning model may process the OCT image data and generate the prediction output. The deep learning model may be, for example, a binary classification model. The OCT image data may be the raw OCT image data generated by an OCT imaging device or may be the preprocessed form of raw OCT image data (e.g., preprocessed via any number of standardization or normalization procedures).

In other embodiments, step 404 includes segmenting, via a segmentation model (e.g., segmentation model 206 in FIGS. 2-3), the OCT image data to form segmented image data. The OCT image data may be the raw OCT image data generated by an OCT imaging device or may be the preprocessed form of raw OCT image data (e.g., preprocessed via any number of standardization or normalization procedures). The segmented image may then be processed by the deep learning model to generate the prediction output.

In still other embodiments, step 404 includes the machine learning model includes a feature-based model (e.g., feature-based model 300 in FIG. 3) and the model system may further include a feature extraction model (e.g., feature extraction model 306 in FIG. 3), CNV classifier 308, or both. The feature extraction model may receive segmented image data from the segmentation model and may use the segmented image data to extract retinal feature data (e.g., retinal feature data 120 in FIGS. 1 and 3) from the segmented image data. The retinal feature data may include at least one of a first feature value related to at least one retinal layer element or a second feature value related to at least one retinal pathological element. The retinal feature data may include, for example, 1 to 200 retinal features (or values for retinal features).

The machine learning model in step 404 may also be used to process clinical data (e.g., clinical data 122 in FIGS. 1-3) in addition to OCT image data, segmented image data, or retinal feature data. The clinical data may include baseline clinical data. For example, the clinical data may include a baseline choroidal neovascularization (CNV) type, a baseline visual acuity measurement, or a baseline age. The baseline visual acuity measurement may be a baseline BCVA or some other type of visual acuity measurement.

When the machine learning model includes a deep learning model for processing either OCT image data or segmented image data, the deep learning model may include, for example, a convolutional neural network (CNN) system, which may include ResNet-50 or a modified form of ResNet-50. In one or more embodiments, a first portion of the deep learning system (e.g., ResNet-50 without one or more top layers) is used to process the OCT image data or the segmented image data to generate a first intermediate output. A second portion of the deep learning model (e.g., the replacement for the one or more top layers of ResNet-50) may include a custom dense portion (e.g., one or more dense layers). A set of vectors for the one or more clinical variables included in the clinical data may be concatenated to the first intermediate output to form a second intermediate output. The second intermediate output may be processed using the second portion of the deep learning model, the custom dense layer portion, to generate the prediction output.

Step 406 includes generating a final output that indicates a risk of developing fibrosis in the retina based on the prediction output. The final output, which may be, for example, final output 114 in FIGS. 1-3, may include the prediction output and/or a binary classification formed based on the prediction output. In some cases, the final output, which may be final output 114 in FIGS. 1-3, may be a report that includes other information in addition to the prediction output and/or binary classification. For example, this other information may include a clinical trial recommendation to either include or exclude the subject from a clinical trial based on either the prediction output or the binary classification. The information may include a treatment recommendation to change a type of treatment, adjust a treatment regimen for the subject, or both based on either the prediction output or the binary classification. In some embodiments, the final output includes at least a portion of the input used to generate prediction output.

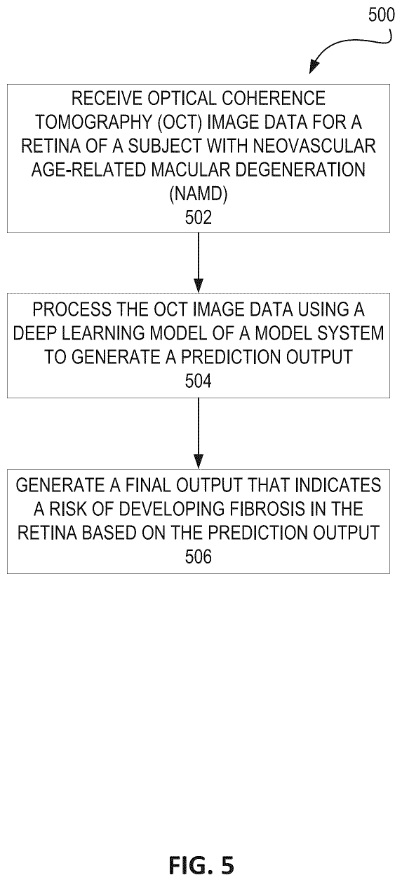

FIG. 5 is a flowchart of a process 500 for predicting fibrosis development using OCT image data in accordance with one or more embodiments. In one or more embodiments, process 500 may be implemented using prediction system 100 described in FIG. 1 and/or fibrosis predictor 110 described in FIGS. 1-2. Process 500 includes various steps and may be described with continuing reference to FIGS. 1-2. One or more steps that are not expressly illustrated in FIG. 5 may be included before, after, in between, or as part of the steps of process 500. In some embodiments, process 500 may begin with step 502. Process 500 in FIG. 5 may be a more detailed version of process 400 in FIG. 4 specific to the generation of a final output based on OCT image data.

Step 502 includes receiving optical coherence tomography (OCT) image data for a retina of a subject with neovascular age-related macular degeneration (nAMD). The OCT image data may be, for example, OCT image data 116 described with respect to FIGS. 1-2. The OCT image data may be the raw OCT image data generated by an OCT imaging device or may be the preprocessed form of raw OCT image data (e.g., preprocessed via any number of standardization or normalization procedures).

Step 504 includes processing the OCT image data using a deep learning model of a model system to generate a prediction output. The deep learning model may be, for example, deep learning model 200 in FIG. 2. In one or more embodiments, the deep learning model includes a binary classification model. The deep learning model may include a convolutional neural network.

The prediction output generated in step 504 may be, for example, prediction output 204 in FIG. 2. The prediction output may be the likelihood that the retina of a subject diagnosed with nAMD will develop fibrosis. In one or more embodiments, the prediction output is a binary classification that indicates whether fibrosis development is predicted or not. For example, the binary classification may indicate: a low or high risk for fibrosis development, a positive or negative prediction for fibrosis development, or other type of binary classification. The prediction output may take the form of a score (e.g., a probability distribution value or likelihood value) that is indicative of whether fibrosis development is predicted or not.

Step 506 includes generating a final output that indicates a risk of developing fibrosis in the retina based on the prediction output. The final output may be, for example, final output 114 described with respect to FIGS. 1-2. The final output may be similar to the final output described with respect to step 406 in FIG. 4.

In some embodiments, step 502 includes receiving clinical data (e.g., clinical data 122 in FIGS. 1 and 2 for processing. The clinical data may include at least one of a baseline CNV type, a baseline visual acuity measurement, or a baseline age. In these embodiments, when clinical data is received in step 502, step 504 may include processing both the OCT image data and the clinical data using the model system to generate the prediction output.

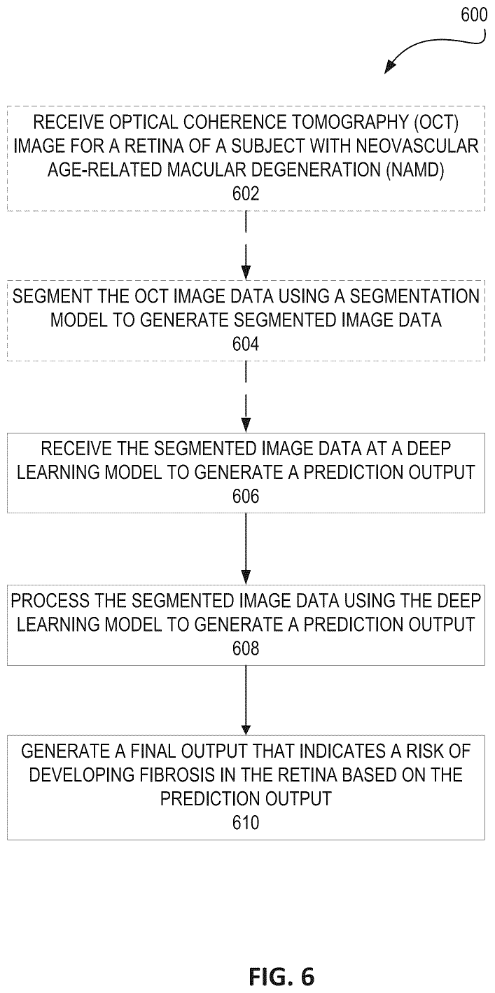

FIG. 6 is a flowchart of a process 600 for predicting fibrosis development in accordance with one or more embodiments. In one or more embodiments, process 600 may be implemented using prediction system 100 described in FIG. 1 and/or fibrosis predictor 110 described in FIGS. 1-2. Process 600 includes various steps and may be described with continuing reference to FIGS. 1-2. One or more steps that are not expressly illustrated in FIG. 6 may be included before, after, in between, or as part of the steps of process 600. In some embodiments, process 600 may begin with step 602. Process 600 in FIG. 6 may be a more detailed version of process 400 in FIG. 4 specific to the generation of a final output based on segmented image data.

Step 602 may optionally include receiving optical coherence tomography (OCT) image for a retina of a subject with neovascular age-related macular degeneration (nAMD). The OCT image data may be, for example, OCT image data 116 described with respect to FIGS. 1-2. The OCT image data may be the raw OCT image data generated by an OCT imaging device or may be the preprocessed form of raw OCT image data (e.g., preprocessed via any number of standardization or normalization procedures).

Step 604 may optionally include segmenting the OCT image data using a segmentation model to generate segmented image data. The segmentation model may be, for example, segmentation model 206 in FIG. 2. In one or more embodiments, the segmentation model comprises a U-Net-based architecture that is pretrained on training OCT image data comprising OCT images annotated via human graders (e.g., certified graders). The segmentation model may be trained to automatically segment one or more retinal pathological elements (e.g., SHRM, SRF, PED, IRF, etc.), one or more retinal layer elements (e.g., ILM, OPL-HFL, RPE, BM, etc.), or both.

Step 606 may include receiving the segmented image data at a deep learning model. The deep learning model may be, for example, deep learning model 200 in FIG. 2.

Step 608 may include processing the segmented image data using the deep learning model to generate a prediction output (e.g., prediction output 204 in FIG. 2). The prediction output may be the likelihood that the retina of a subject diagnosed with nAMD will develop fibrosis. In one or more embodiments, the prediction output is a binary classification that indicates whether fibrosis development is predicted or not. For example, the binary classification may indicate: a low or high risk for fibrosis development, a positive or negative prediction for fibrosis development, or other type of binary classification. The prediction output may take the form of a score (e.g., a probability distribution value or likelihood value) that is indicative of whether fibrosis development is predicted or not.

Step 610 may include generating a final output that indicates a risk of developing fibrosis in the retina based on the prediction output. The final output may be, for example, final output 114 described with respect to FIGS. 1-2. The final output may be similar to the final output described with respect to step 406 in FIG. 4.

In some embodiments, step 602 includes receiving clinical data (e.g., clinical data 122 in FIGS. 1 and 2 for processing. The clinical data may include at least one of a baseline CNV type, a baseline visual acuity measurement, or a baseline age. In these embodiments, when clinical data is received in step 602, step 608 may include processing both the segmented image data and the clinical data using the deep learning model to generate the prediction output.

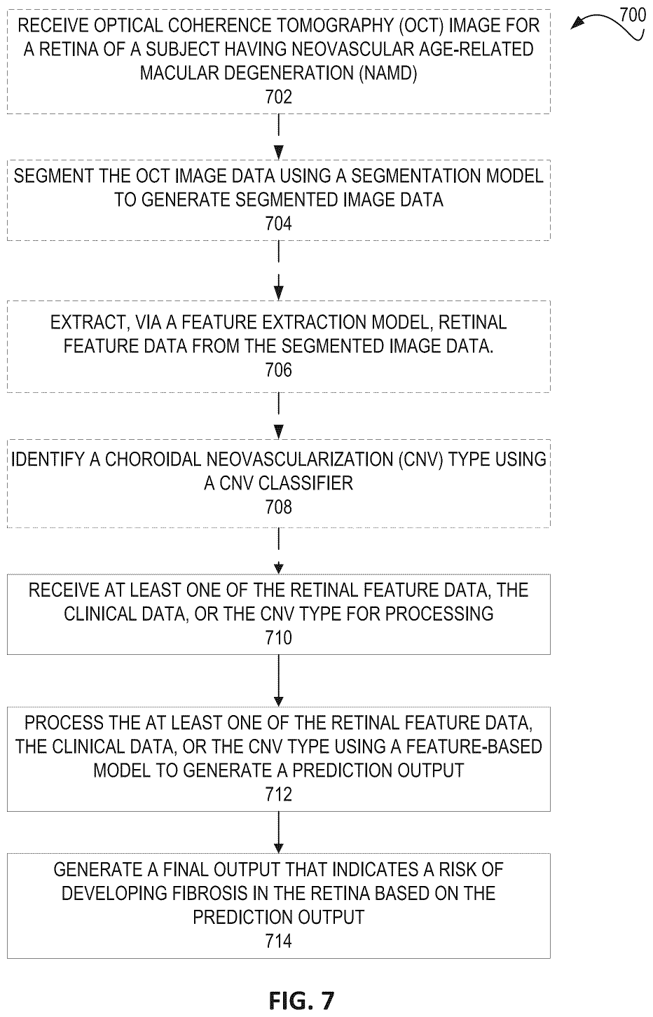

FIG. 7 is a flowchart of a process 700 for predicting fibrosis development in accordance with one or more embodiments. In one or more embodiments, process 700 may be implemented using prediction system 100 described in FIG. 1 and/or fibrosis predictor 110 described in FIGS. 1 and 3. Process 700 includes various steps and may be described with continuing reference to FIGS. 1 and 3. One or more steps that are not expressly illustrated in FIG. 7 may be included before, after, in between, or as part of the steps of process 700. In some embodiments, process 700 may begin with step 702. Process 700 in FIG. 7 may be a more detailed version of process 400 in FIG. 4 specific to the generation of a final output using a feature-based model.

Step 702 may optionally include receiving optical coherence tomography (OCT) image for a retina of a subject with neovascular age-related macular degeneration (nAMD). The OCT image data may be, for example, OCT image data 116 described with respect to FIGS. 1 and 3. The OCT image data may be the raw OCT image data generated by an OCT imaging device or may be the preprocessed form of raw OCT image data (e.g., preprocessed via any number of standardization or normalization procedures).

Step 704 may optionally include segmenting the OCT image data using a segmentation model to generate segmented image data (e.g., segmented image data 118 in FIGS. 1 and 3). The segmentation model may be, for example, segmentation model 206 in FIG. 3. In one or more embodiments, the segmentation model comprises a U-Net-based architecture that is pretrained on training OCT image data comprising OCT images annotated via human graders (e.g., certified graders). The segmentation model may be trained to automatically segment one or more retinal pathological elements (e.g., SHRM, SRF, PED, IRF, etc.), one or more retinal layer elements (e.g., ILM, OPL-HFL, RPE, BM, etc.), or both.

Step 706 optionally includes extracting, via a feature extraction model, retinal feature data from the segmented image data. The feature extraction model may be, for example, feature extraction model 306 in FIG. 3. The feature extraction model may receive the segmented image data from the segmentation model and may use the segmented image data to extract retinal feature data (e.g., retinal feature data 120 in FIGS. 1 and 3) from the segmented image data. The retinal feature data may include at least one of a first feature value related to at least one retinal layer element or a second feature value related to at least one retinal pathological element. The retinal feature data may include, for example, 1 to 200 retinal features (or values for retinal features).

Step 708 may optionally include identifying a choroidal neovascularization (CNV) type using a CNV classifier (e.g., CNV classifier 308). The CNV classifier may be implemented using, for example, without limitation, a deep learning model that uses OCT image data to detect and identify CNV type. This CNV type may be a model-generated CNV type, which may be distinct from a baseline CNV type included in clinical data (e.g., where the CNV type is determined by human graders based on FA image data).

Step 710 includes receiving at least one of the retinal feature data, clinical data, or the CNV type for processing. The CNV type in step 710 may be the model-generated CNV type identified in step 708. The retinal feature data may be the retinal feature data generated in step 706. The clinical data may be, for example, clinical data 122 in FIGS. 1 and 3. The clinical data may include at least one of a baseline CNV type, a baseline visual acuity measurement, or a baseline age.

Step 712 includes processing the at least one of the clinical data, the retinal feature data, or the CNV type using a feature-based model to generate a prediction output. The feature-based model may be, for example, feature-based model 300 in FIG. 3. The feature-based model may include, for example, a regression model. The CNV type in step 708 may be the model-generated CNV type identified in step 708.

The prediction output may be the likelihood that the retina of a subject diagnosed with nAMD will develop fibrosis. In one or more embodiments, the prediction output is a binary classification that indicates whether fibrosis development is predicted or not. For example, the binary classification may indicate: a low or high risk for fibrosis development, a positive or negative prediction for fibrosis development, or other type of binary classification. The prediction output may take the form of a score (e.g., a probability distribution value or likelihood value) that is indicative of whether fibrosis development is predicted or not.

Step 714 includes generating a final output that indicates a risk of developing fibrosis in the retina based on the prediction output. The final output may be, for example, final output 114 described with respect to FIGS. 1 and 3. The final output may be similar to the final output described with respect to step 406 in FIG. 4.

### IV. Exemplary Images

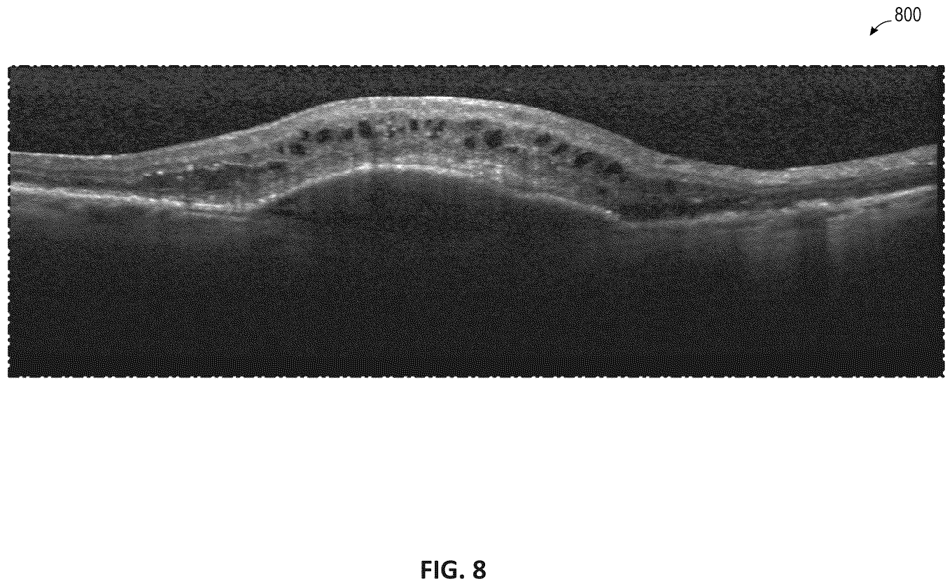

FIG. 8 is an OCT image in accordance with one or more embodiments. OCT image 800 is one example of an implementation for an OCT image that may be included in OCT image data 116 described above in Sections II.A. and II.B. OCT image 800 may be a single OCT B-scan. In one or more embodiments, OCT image 800 may be processed as part of model input 202 for deep learning model 200 in FIG. 2. In one or more embodiments,

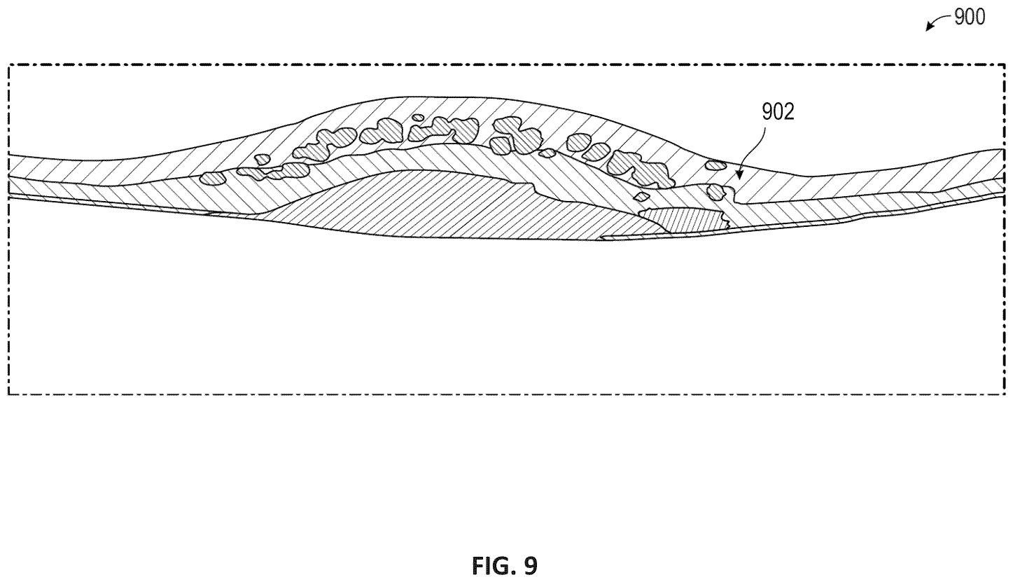

FIG. 9 is a segmented image in accordance with one or more embodiments. Segmented image 900 is example of an implementation for a segmented image that may be included in segmented image data 118 described above in Sections II.A. and II.B. Segmented image 900 may be a representation of an OCT image (e.g., OCT image 800 in FIG. 8) in which a plurality of masks 902 have been overlaid on the representation. In other examples, segmented image 900 may be an OCT image (e.g., OCT image 800 in FIG. 8) over which the plurality of masks 902 have been overlaid on the representation.

Here, plurality of masks 902 represent various retinal elements. These retinal elements may include, but are not limited to, intraretinal fluid (IRF), subretinal fluid (SRF), subretinal hyperreflective material (SHRM), pigment epithelial detachment (PED), an interface between (may be inclusive of) the inner limiting membrane and (ILM) layer and external limiting membrane (ELM) layer, an interface between (may be inclusive of) the ILM layer and a retinal pigment epithelial (RPE) layer, and an interface between (may be inclusive of) the RPE layer and Bruch's membrane (BM) layer.

### V. Exemplary Training and Validation of Machine Learning Models

### V.A. Exemplary Data

Various machine learning models were trained and their performance evaluated. Training including using training data obtained from and/or generated based on data obtained from a clinical trial. In particular, 935 eyes were selected from the eyes of 1097 treatment-naïve eyes of nAMD subjects who participated in phase 3 of the randomized, multicenter HARBOR trial. These nAMD subjects were treated with ranibizumab 0.5 or 2.0 mg on a monthly or as-needed basis over 12 months. In the HARBOR trial, CNV type was graded based on FA images as occult CNV (e.g., with occult CNV lesions), predominantly classic CNV, or minimally classic CNV. In the HARBOR trial, fibrosis presence was assessed at day 0, month 3, month 6, month 12, and month 24.

The 935 eyes selected included those for which unambiguous fibrosis records were available at month 12 and for which baseline OCT image data was available. The OCT image data comprised a baseline OCT volume scan for each eye.

For training of the deep learning models, five equally-spaced B-scans were selected from each of the 935 OCT volume scans, covering 1.44 mm of the central macula. Specifically, out of the 128 B-scans, scans 49, 56, 63, 70, and 77 were selected. A first deep learning model was trained using the raw OCT B-scans. A second deep learning model was trained using the segmented images generated based on the raw OCT B-scans. The data was augmented using random horizontal and vertical flips, scaling, rotation, and shearing to yield a total of 30,000 samples.

The OCT volume scans were segmented using a pretrained segmentation model (e.g., one example of an implementation for segmentation model 206 in FIGS. 2-3). The segmentation model was pretrained based on annotations made by certified graders. The segmentation model was trained to automatically segment 4 retinal pathological elements (SHRM, SRF, PED, and IRF) along with 5 retinal layer elements (ILM, OPL-HLF interface, inner and outer boundaries of RPE, and BM). The elements were segmented in three topographic locations (e.g., 1 mm, 3 mm, and 6 mm diameter circle) per OCT volume scan.

Based on the segmented image data, retinal feature data was extracted using a feature extraction model (e.g., one example of an implementation for feature extraction model 306 in FIG. 3). The feature extraction model automatically extracted 105 quantitative retinal features. Specifically, these retinal features include 36 volumetric, pathology-related features (e.g., 4 retinal pathological elements for each of 3 readout variants for each of 3 topographic locations), 15 layer-related volume features (e.g., 5 pairs of layers for each of 3 topographic locations), and 54 layer-related thickness features (e.g., 6 pairs of layers for each of 3 readout variants for each 3 topographic locations). All features were derived for each individual B-scan of the OCT volume scan and then combined to form volume-wide measurements.

### V.B. Training of Machine Learning Models

Presence of fibrosis at month 12 was defined as the outcome for training and validating the models. Folds were predefined for five-fold cross validation on the level of subject numbers to ensure that the outcome variable was stratified across folds. This was repeated ten times, resulting in 10 repeats with 5 splits to yield a total of 50 train/test splits. A model was always trained on a training set, then used to predict the test set. Validation was done for all 50 splits for the feature-based models (e.g., examples of implementations for feature-based model 300 in FIG. 3), whereas validation was only done for the five splits of the first repeat for the deep learning models (e.g., examples of implementations for deep learning model 200 in FIG. 2) in order to limit computational effort.

For the feature-based models, Lasso regularization was used for fitting the feature-based models (e.g., logistic regression models) with various configurations of features. Combinations of selected OCT-derived quantitative retinal features and three baseline clinical variables (CNV type, BCVA and, age) were used. The degree of regularization was set to a constant high value when OCT-derived quantitative retinal features were used.

For the deep learning models (e.g., convolutional neural networks), a ResNet-50 architecture pretrained on ImageNet was used. The architecture was either adjusted by replacing the top layers with a custom dense part, allowing concatenation of the vectors of clinical variables to the OCT image data, or was used as is when not using clinical data. Twenty epochs of transfer learning keeping the base ResNet-50 layers frozen were applied, followed by 40 or 120 epochs for fine tuning the complete network on the segmented image data or raw OCT image data, both when using and not using clinical data.

### V.C. Model Performance

For a baseline comparison, feature-based models were built for clinical data only (e.g., one for baseline CNV type only; one for baseline visual acuity (BVA) and age only; and one for baseline CNV, BVA, and age). Performance was evaluated using the area under the receiver operating characteristic (AUC) curve by plotting the observed event rate against the predicted event rate. Additionally, Youden's index was applied to AUC curves to select the cutoff points, and reported as the positive and negative predictive values of the models' prediction. Specificity and sensitivity were also evaluated.

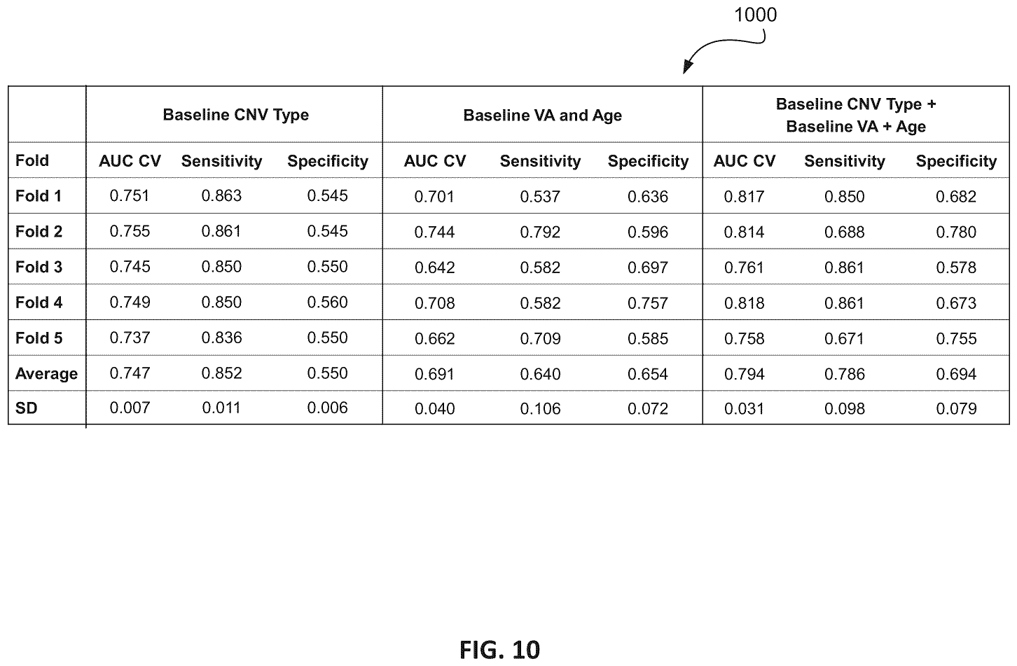

FIG. 10 is a table 1000 comparing the statistical results for feature-based models that use clinical data in accordance with one or more embodiments. As shown in table 1000, based on average AUC, the feature-based model using baseline CNV type alone and the feature-based model using baseline CNV type along with BVA and age performed best. However, the feature-based model using baseline CNV alone had a lower specificity than the feature-based model using baseline CNV type with BVA and age.

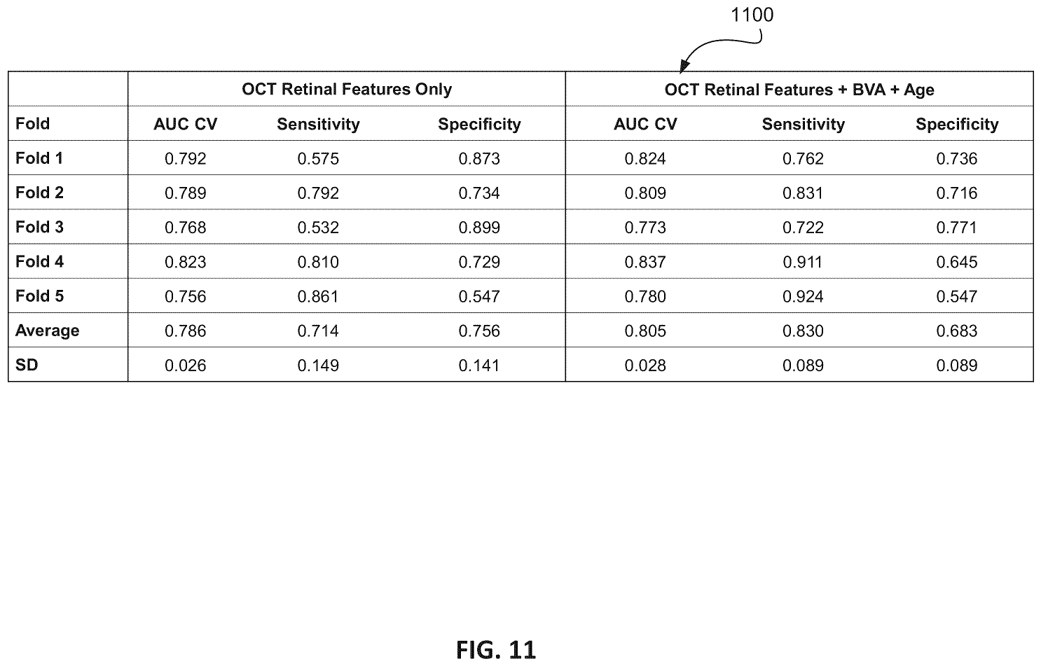

FIG. 11 is a table 1100 comparing the statistical results for feature-based models using retinal features derived from OCT image data in accordance with one or more embodiments. As shown in table 1100, based on average AUC, a first feature-based model using OCT-derived retinal features to predict fibrosis development and a second feature-based model using OCT-derived retinal features along with BVA and age to predict fibrosis development performed similarly as compared to feature-based models using baseline CNV type (as shown in FIG. 10). Although not shown in table 1100, adding baseline CNV type to the first or second feature-based models increased average AUC to 0.809 and 0.821, respectively. These results show that a feature-based model using OCT-derived retinal features may be used to accurately and reliably predict fibrosis development.

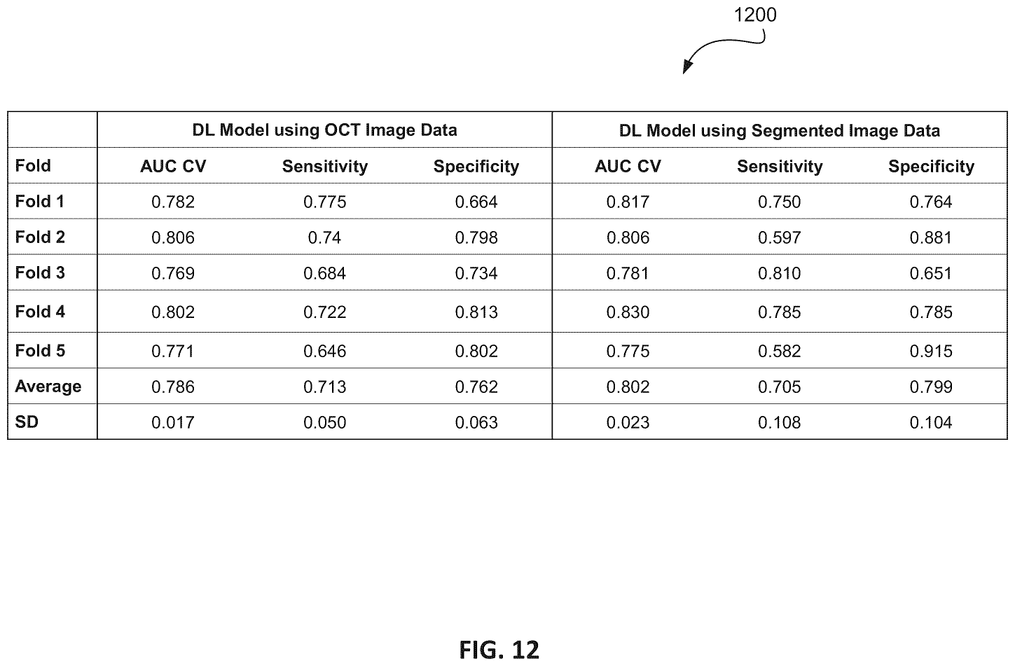

FIG. 12 is a table 1200 comparing the statistical results for deep learning models using OCT image data and segmented image data in accordance with one or more embodiments. As depicted in table 1200, the average AUC for a deep learning model using segmented images to predict fibrosis development was slightly higher than the average AUC for a deep learning model using OCT image data to predict fibrosis development. Further, the average AUCs for these two deep learning models performed similarly as compared to the feature-based models using baseline CNV type (as shown in FIG. 10).

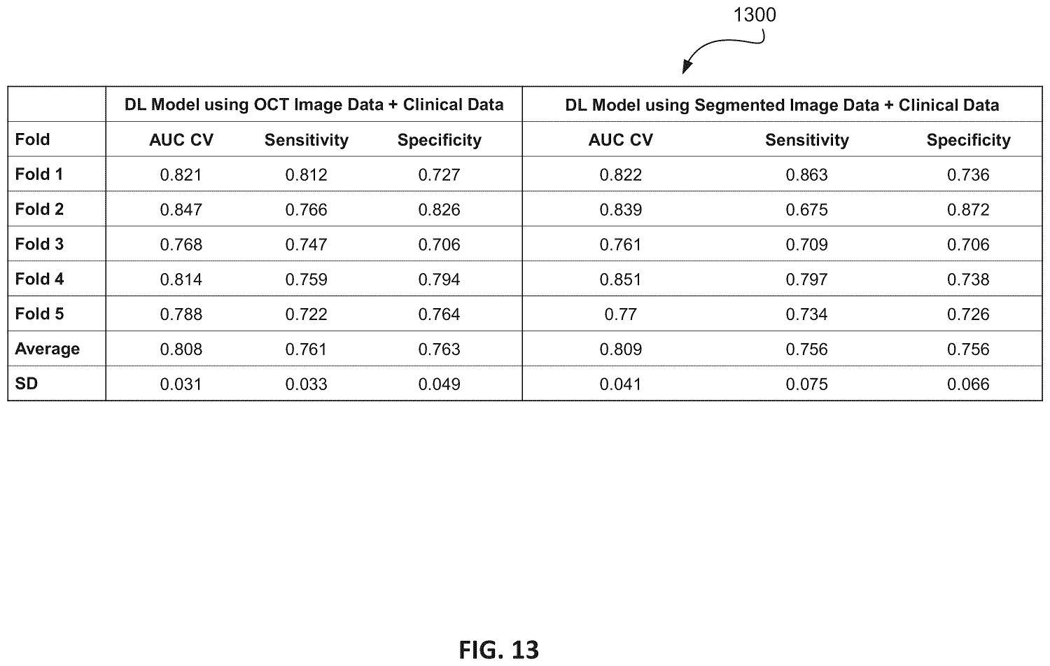

FIG. 13 is a table 1300 comparing the statistical results for deep learning models using OCT image data and segmented image data in combination with clinical data in accordance with one or more embodiments. As depicted in table 1300, adding clinical data (e.g., BVA, age, and baseline CNV type) to the deep learning models increased the average AUC for the deep learning model using segmented image data more than the average AUC for deep learning model using OCT image data.

### VI. Computer-Implemented System

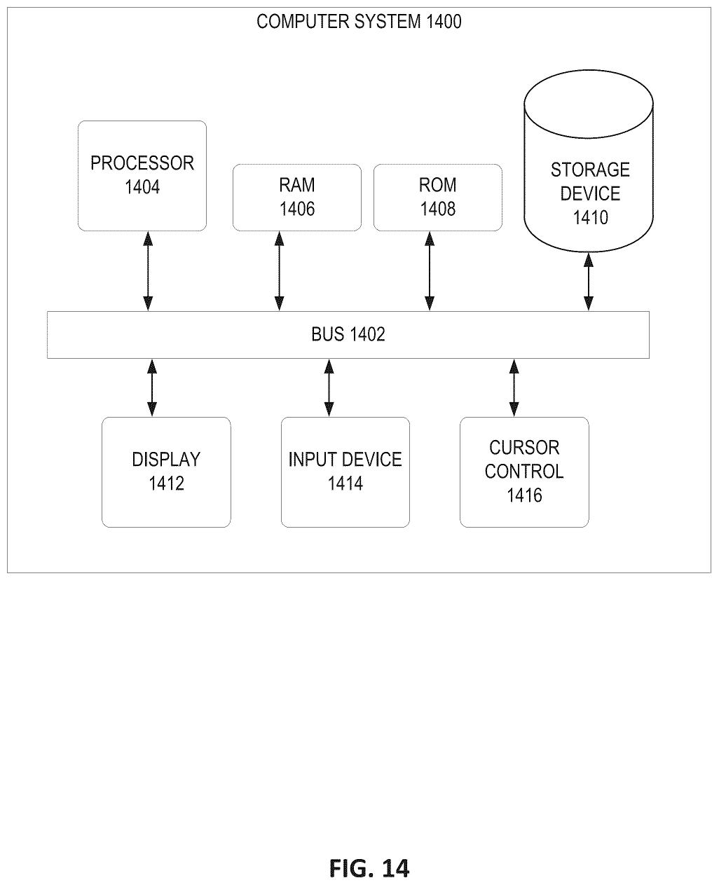

FIG. 14 is a block diagram that illustrates a computer system, in accordance with various embodiments. Computer system 1400 may be one example of an implementation for computing platform 102 in FIG. 1. In various embodiments of the present teachings, computer system 1400 can include a bus 1402 or other communication mechanism for communicating information, and a processor 1404 coupled with bus 1402 for processing information. In various embodiments, computer system 1400 can also include a memory, which can be a random access memory (RAM) 1406 or other dynamic storage device, coupled to bus 1402 for determining instructions to be executed by processor 1404. Memory also can be used for storing temporary variables or other intermediate information during execution of instructions to be executed by processor 1404. In various embodiments, computer system 1400 can further include a read only memory (ROM) 1408 or other static storage device coupled to bus 1402 for storing static information and instructions for processor 1404. A storage device 1410, such as a magnetic disk or optical disk, can be provided and coupled to bus 1402 for storing information and instructions.

In various embodiments, computer system 1400 can be coupled via bus 1402 to a display 1412, such as a cathode ray tube (CRT) or liquid crystal display (LCD), for displaying information to a computer user. An input device 1414, including alphanumeric and other keys, can be coupled to bus 1402 for communicating information and command selections to processor 1404. Another type of user input device is a cursor control 1416, such as a mouse, a trackball or cursor direction keys for communicating direction information and command selections to processor 1404 and for controlling cursor movement on display 1412. This input device 1414 typically has two degrees of freedom in two axes, a first axis (i.e., x) and a second axis (i.e., y), that allows the device to specify positions in a plane. However, it should be understood that input devices 1414 allowing for 3 dimensional (x, y and z) cursor movement are also contemplated herein.

Consistent with certain implementations of the present teachings, results can be provided by computer system 1400 in response to processor 1404 executing one or more sequences of one or more instructions contained in RAM 1406. Such instructions can be read into RAM 1406 from another computer-readable medium or computer-readable storage medium, such as storage device 1410. Execution of the sequences of instructions contained in RAM 1406 can cause processor 1404 to perform the processes described herein. Alternatively hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 1404 for execution. Such a medium can take many forms, including but not limited to, non-volatile media, volatile media, and transmission media. Examples of non-volatile media can include, but are not limited to, optical, solid state, magnetic disks, such as storage device 1410. Examples of volatile media can include, but are not limited to, dynamic memory, such as RAM 1406. Examples of transmission media can include, but are not limited to, coaxial cables, copper wire, and fiber optics, including the wires that comprise bus 1402.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 1404 of computer system 1400 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein, including flow charts, diagrams and accompanying disclosure, can be implemented using computer system 1400 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 1400, whereby processor 1404 would execute the analyses and determinations provided by these engines, subject to instructions provided by any one of, or a combination of, memory components RAM 1406, ROM 1408, or storage device 1410 and user input provided via input device 1414.

### VII. Exemplary Definitions and Context

The disclosure is not limited to the exemplary embodiments and applications described herein or to the manner in which the exemplary embodiments and applications operate or are described herein. Moreover, the figures may show simplified or partial views, and the dimensions of elements in the figures may be exaggerated or otherwise not in proportion.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology and toxicology are described herein are those well-known and commonly used in the art.

In addition, as the terms “on,” “attached to,” “connected to,” “coupled to,” or similar words are used herein, one element (e.g., a component, a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

The term “subject” may refer to a subject of a clinical trial, a person undergoing treatment, a person undergoing anti-cancer therapies, a person being monitored for remission or recovery, a person undergoing a preventative health analysis (e.g., due to their medical history), or any other person or patient of interest. In various cases, “subject” and “patient” may be used interchangeably herein.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

As used herein, the term “about” used with respect to numerical values or parameters or characteristics that can be expressed as numerical values means within ten percent of the numerical values. For example, “about 50” means a value in the range from 45 to 55, inclusive.

The term “ones” means more than one.

As used herein, the term “plurality” can be 2, 3, 4, 5, 6, 7, 8, 9, 10, or more. As used herein, the term “set of” means one or more. For example, a set of items includes one or more items.

As used herein, the phrase “at least one of,” when used with a list of items, means different combinations of one or more of the listed items may be used and only one of the items in the list may be used. The item may be a particular object, thing, step, operation, process, or category. In other words, “at least one of” means any combination of items or number of items may be used from the list, but not all of the items in the list may be used. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, a “model” may include one or more algorithms, one or more mathematical techniques, one or more machine learning algorithms, or a combination thereof.

As used herein, “machine learning” may include the practice of using algorithms to parse data, learn from the data, and then make a determination or prediction about something in the world. Machine learning may use algorithms that can learn from data without relying on rules-based programming. Deep learning may be one form of machine learning.

As used herein, an “artificial neural network” or “neural network” (NN) may refer to mathematical algorithms or computational models that mimic an interconnected group of artificial neurons that processes information based on a connectionist approach to computation. Neural networks, which may also be referred to as neural nets, can employ one or more layers of nonlinear units to predict an output for a received input. Some neural networks may include one or more hidden layers in addition to an output layer. The output of each hidden layer may be used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in two ways; when it is being trained it is in training mode and when it puts what it has learned into practice it is in inference (or prediction) mode. Neural networks may learn through a feedback process (e.g., backpropagation) which allows the network to adjust the weight factors (modifying its behavior) of the individual nodes in the intermediate hidden layers so that the output matches the outputs of the training data. In other words, a neural network may learn by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), a U-Net, a fully convolutional network (FCN), a stacked FCN, a stacked FCN with multi-channel learning, a Squeeze and Excitation embedded neural network, a MobileNet, or another type of neural network.

As used herein, “deep learning” may refer to the use of multi-layered artificial neural networks to automatically learn representations from input data such as images, video, text, etc., without human provided knowledge, to deliver highly accurate predictions in tasks such as object detection/identification, speech recognition, language translation, etc.

### VIII. Recitation of Exemplary Embodiments

Embodiment 1: A method, comprising: receiving optical coherence tomography (OCT) image data for a retina of a subject with neovascular age-related macular degeneration (nAMD); processing the OCT image data using a model system comprising a machine learning model to generate a prediction output; and generating a final output that indicates a risk of developing fibrosis in the retina based on the prediction output.

Embodiment 2: The method of embodiment 1, wherein the machine learning model comprises a deep learning model and wherein the processing comprises: segmenting, via a segmentation model comprising at least one neural network, the OCT image data to form segmented image data; and processing the segmented image data using the deep learning model of the model system to generate the prediction output.

Embodiment 3: The method of embodiment 2, wherein the machine learning model comprises a regression model and wherein the processing further comprises: extracting, via a feature extraction model, retinal feature data from the segmented image data, wherein the retinal feature data comprises at least one of a first feature value related to at least one retinal layer element or a second feature value related to at least one retinal pathological element; and processing the OCT image data using the regression model to generate the prediction output.

Embodiment 4: The method of any one of embodiments 1-3, wherein the machine learning model comprises at least one convolutional neural network.

Embodiment 5: The method of any one of embodiments 1-4, wherein the machine learning model comprises a deep learning model and wherein the processing comprises: processing the OCT image data and clinical data using the deep learning model to generate the prediction output, wherein the clinical data comprises at least one of a baseline choroidal neovascularization (CNV) type, a baseline visual acuity measurement, or a baseline age.

Embodiment 6: The method of embodiment 5, wherein the deep learning model comprises a convolutional neural network (CNN) system in which a first portion of the CNN system comprises a convolutional neural network and a second portion of the CNN system comprises a custom dense layer portion and wherein the processing of the OCT image data and the clinical data comprises: processing the OCT image data using the first portion of the CNN system to generate a first intermediate output; concatenating a set of vectors for the clinical data to the first intermediate output to form a second intermediate output; and processing the second intermediate output using the custom dense layer portion to generate the prediction output.

Embodiment 7: The method of any one of embodiments 1-6, wherein the final output comprises at least one of: a binary classification indicating whether fibrosis development is predicted; a clinical trial recommendation to either include or exclude the subject from a clinical trial based on either the prediction output or the binary classification; or a treatment recommendation to at least one of change a type of treatment or adjust a treatment regimen for the subject based on either the prediction output or the binary classification.

Embodiment 8: A method, comprising: receiving optical coherence tomography (OCT) image data for a retina of a subject with neovascular age-related macular degeneration (nAMD); segmenting the OCT image data using a segmentation model to generate segmented image data; processing the segmented image data using a deep learning model to generate a prediction output; and generating a final output that indicates a risk of developing fibrosis in the retina based on the prediction output.

Embodiment 9: The method of embodiment 8, wherein at least one of the segmentation model or the deep learning model comprises at least one convolutional neural network.

Embodiment 10: The method of embodiment 8 or embodiment 9, wherein the processing comprises: processing the segmented image data and clinical data using the deep learning model to generate the prediction output, wherein the clinical data comprises at least one of a baseline choroidal neovascularization (CNV) type, a baseline visual acuity measurement, or a baseline age.

Embodiment 11: The method of embodiment 10, wherein the deep learning model comprises a convolutional neural network (CNN) system in which a first portion of the CNN system comprises a convolutional neural network and a second portion of the CNN system comprises a custom dense layer portion and wherein the processing of the segmented image data and the clinical data comprises: processing the segmented image data using the first portion of the CNN system to generate a first intermediate output; concatenating a set of vectors for the clinical data to the first intermediate output to form a second intermediate output; and processing the second intermediate output using the custom dense layer portion to generate the prediction output.

Embodiment 12: The method of any one of embodiments 8-11, wherein the final output comprises at least one of: a binary classification indicating whether fibrosis development is predicted; a clinical trial recommendation to either include or exclude the subject from a clinical trial based on either the prediction output or the binary classification; or a treatment recommendation to at least one of change a type of treatment or adjust a treatment regimen for the subject based on either the prediction output or the binary classification.

Embodiment 13: A method comprising: receiving at least one of clinical data or retinal feature data for a retina of a subject with neovascular age-related macular degeneration (nAMD); processing the at least one of the clinical data or the retinal feature data using a regression model to generate a prediction output; and generating a final output that indicates a risk of developing fibrosis in the retina based on the prediction output.

Embodiment 14: The method of embodiment 13, further comprising: extracting, via a feature extraction model, the retinal feature data from segmented image data.

Embodiment 15: The method of embodiment 14, further comprising: segmenting, via a segmentation model comprising at least one neural network, OCT image data to form the segmented image data.

Embodiment 16: The method of any one of embodiments 13-15, wherein the clinical data comprises at least one of a baseline choroidal neovascularization (CNV) type, a baseline visual acuity measurement, or a baseline age and wherein the retinal feature data comprises at least one of a first feature value related to at least one retinal layer element or a second feature value related to at least one retinal pathological element.

Embodiment 17: The method of any one of embodiments 13-16, wherein the regression model is trained using at least one of Ridge regularization, Lasso regularization, or Elastic Net regularization.

Embodiment 18: The method of any one of embodiments 13-17, wherein the prediction output comprises a score that indicates a probability that fibrosis is likely to develop.

Embodiment 19: The method of any one of embodiments 13-18, wherein the final output comprises at least one of: a binary classification indicating whether fibrosis development is predicted; a clinical trial recommendation to either include or exclude the subject from a clinical trial based on either the prediction output or the binary classification; or a treatment recommendation to at least one of change a type of treatment or adjust a treatment regimen for the subject based on either the prediction output or the binary classification.

Embodiment 20: The method of any one of embodiment 3 or embodiments 13-19, wherein the retinal feature data comprises at least one of a grade for subretinal hyperreflective material (SRHM), a grade for pigment epithelial detachment (PED), a maximal height of subretinal fluid (SRF), a maximal thickness between an interface of outer plexiform layer (OPL) and Henle's fiber layer (HFL) and a retinal pigment epithelial (RPE) layer, or a thickness of between an inner limiting membrane (ILM) layer to the RPE layer.

### IX. Additional Considerations

The headers and subheaders between sections and subsections of this document are included solely for improving readability and do not imply that features cannot be combined across sections and subsection. Accordingly, sections and subsections do not describe separate embodiments. Any one or more of the embodiments described herein in any section or with respect to any FIG. may be combined with or otherwise integrated with any one or more of the other embodiments described herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, although the present invention as claimed has been specifically disclosed by embodiments and optional features, it should be understood that modification and variation of the concepts disclosed herein may be employed by those skilled in the art, and that such modifications and variations are considered to be within the scope of this invention as defined by the appended claims.

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements (e.g., elements in block or schematic diagrams, elements in flow diagrams, etc.) without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail to avoid obscuring the embodiments.

1. A method, comprising:
receiving optical coherence tomography (OCT) image data for a retina of a subject with neovascular age-related macular degeneration (nAMD);
processing the OCT image data using a model system comprising a machine learning model to generate a prediction output; and
generating a final output that indicates a risk of developing fibrosis in the retina based on the prediction output.

2. The method of claim 1, wherein the machine learning model comprises a deep learning model and wherein the processing comprises:
segmenting, via a segmentation model comprising at least one neural network, the OCT image data to form segmented image data; and
processing the segmented image data using the deep learning model of the model system to generate the prediction output.

3. The method of claim 2, wherein the machine learning model comprises a regression model and wherein the processing further comprises:
extracting, via a feature extraction model, retinal feature data from the segmented image data, wherein the retinal feature data comprises at least one of a first feature value related to at least one retinal layer element or a second feature value related to at least one retinal pathological element; and
processing the OCT image data using the regression model to generate the prediction output.

4. The method of claim 1, wherein the machine learning model comprises at least one convolutional neural network.

5. The method of claim 1, wherein the machine learning model comprises a deep learning model and wherein the processing comprises:
processing the OCT image data and clinical data using the deep learning model to generate the prediction output, wherein the clinical data comprises at least one of a baseline choroidal neovascularization (CNV) type, a baseline visual acuity measurement, or a baseline age.

6. The method of claim 5, wherein the deep learning model comprises a convolutional neural network (CNN) system in which a first portion of the CNN system comprises a convolutional neural network and a second portion of the CNN system comprises a custom dense layer portion and wherein the processing of the OCT image data and the clinical data comprises:
processing the OCT image data using the first portion of the CNN system to generate a first intermediate output;
concatenating a set of vectors for the clinical data to the first intermediate output to form a second intermediate output; and
processing the second intermediate output using the custom dense layer portion to generate the prediction output.

7. The method of claim 1, wherein the final output comprises at least one of:
a binary classification indicating whether fibrosis development is predicted;
a clinical trial recommendation to either include or exclude the subject from a clinical trial based on either the prediction output or the binary classification; or
a treatment recommendation to at least one of change a type of treatment or adjust a treatment regimen for the subject based on either the prediction output or the binary classification.

8. A method, comprising:
receiving optical coherence tomography (OCT) image data for a retina of a subject with neovascular age-related macular degeneration (nAMD);
segmenting the OCT image data using a segmentation model to generate segmented image data;
processing the segmented image data using a deep learning model to generate a prediction output; and
generating a final output that indicates a risk of developing fibrosis in the retina based on the prediction output.

9. The method of claim 8, wherein at least one of the segmentation model or the deep learning model comprises at least one convolutional neural network.

10. The method of claim 8, wherein the processing comprises:
processing the segmented image data and clinical data using the deep learning model to generate the prediction output, wherein the clinical data comprises at least one of a baseline choroidal neovascularization (CNV) type, a baseline visual acuity measurement, or a baseline age.

11. The method of claim 10, wherein the deep learning model comprises a convolutional neural network (CNN) system in which a first portion of the CNN system comprises a convolutional neural network and a second portion of the CNN system comprises a custom dense layer portion and wherein the processing of the segmented image data and the clinical data comprises:
processing the segmented image data using the first portion of the CNN system to generate a first intermediate output;
concatenating a set of vectors for the clinical data to the first intermediate output to form a second intermediate output; and
processing the second intermediate output using the custom dense layer portion to generate the prediction output.

12. The method of claim 8, wherein the final output comprises at least one of:
a binary classification indicating whether fibrosis development is predicted;
a clinical trial recommendation to either include or exclude the subject from a clinical trial based on either the prediction output or the binary classification; or
a treatment recommendation to at least one of change a type of treatment or adjust a treatment regimen for the subject based on either the prediction output or the binary classification.

13. A method comprising:
receiving at least one of clinical data or retinal feature data for a retina of a subject with neovascular age-related macular degeneration (nAMD);
processing the at least one of the clinical data or the retinal feature data using a regression model to generate a prediction output; and
generating a final output that indicates a risk of developing fibrosis in the retina based on the prediction output.

14. The method of claim 13, further comprising:
extracting, via a feature extraction model, the retinal feature data from segmented image data.

15. The method of claim 14, further comprising:
segmenting, via a segmentation model comprising at least one neural network, OCT image data to form the segmented image data.

16. The method of claim 13, wherein the clinical data comprises at least one of a baseline choroidal neovascularization (CNV) type, a baseline visual acuity measurement, or a baseline age and wherein the retinal feature data comprises at least one of a first feature value related to at least one retinal layer element or a second feature value related to at least one retinal pathological element.

17. The method of claim 13, wherein the regression model is trained using at least one of Ridge regularization, Lasso regularization, or Elastic Net regularization.

18. The method of claim 13, wherein the prediction output comprises a score that indicates a probability that fibrosis is likely to develop.

19. The method of claim 13, wherein the final output comprises at least one of:
a binary classification indicating whether fibrosis development is predicted;
a clinical trial recommendation to either include or exclude the subject from a clinical trial based on either the prediction output or the binary classification; or
a treatment recommendation to at least one of change a type of treatment or adjust a treatment regimen for the subject based on either the prediction output or the binary classification.

20. The method of claim 13, wherein the retinal feature data comprises at least one of a grade for subretinal hyperreflective material (SRHM), a grade for pigment epithelial detachment (PED), a maximal height of subretinal fluid (SRF), a maximal thickness between an interface of outer plexiform layer (OPL) and Henle's fiber layer (HFL) and a retinal pigment epithelial (RPE) layer, or a thickness of between an inner limiting membrane (ILM) layer to the RPE layer.
